(19) United States
(12) Patent Application Publication
(10) Pub. No.: US 2024/0238019 A1
(43) Pub. Date: Jul. 18, 2024

(54) MEDICAL IMPLANT FOR OSTEOSYNTHESIS

(71) Applicant: Rheinisch-Westfälische Technische Hochschule Aachen, abgekürzt RWTH, Aachen (DE)

(72) Inventors: Hatem ALABDULRAHMAN, Würselen (DE); Johannes GREVEN, Mönchengladbach (DE); Klemens HORST, Aachen (DE); Jörg ESCHWEILER, Heimbach-Vlatten (DE)

(21) Appl. No.: 18/289,560

(22) PCT Filed: Apr. 26, 2022
(86) PCT No.: PCT/EP2022/061066

§ 371 Date: Nov. 3, 2023

(30) Foreign Application Priority Data
May 5, 2021 (DE) 10 2021 111 653.8

(51) Int. Cl. A61B 17/80; A61F 2/30

(52) U.S. Cl. CPC A61B 17/8066; A61F 2/30; A61F 2002/30538

(57) ABSTRACT

A medical implant for osteosynthesis of symphysis ruptures on the human pelvis are described herein. The medical implant comprises a cranial plate implemented for being attached from a cranial side to a first pubic bone and a second pubic bone of a patient to be treated in order to connect the pubic bones, a posterior plate implemented for being attached from a posterior side to the first pubic bone and the second pubic bone of the patient in order to connect the pubic bones, wherein the cranial plate and the posterior plate are connected to each other by means of at least one first connecting arm. A method for osteosynthesis of a symphysis rupture on a training body is also described herein.

The invention relates to a medical implant for osteosynthesis of symphysis ruptures on the human pelvis, comprising a cranial plate implemented for being attached from a cranial side to a first pubic bone and a second pubic bone of a patient to be treated in order to connect the pubic bones. The invention further relates to a method for osteosynthesis of a symphysis rupture on a training body for training purposes.

In recent decades, steady advances have been made in various operative techniques and the treatment of bone fractures using implants, such as intramedullary nails, or osteosynthesis methods using plates. Nevertheless, there are areas in which ongoing development has stagnated.

The injury characteristics of the polytraumatized patient, particularly fractures of the pelvic ring, present great challenges still today for trauma surgery treatment, even though treatment has improved steadily in recent decades. Many treatment strategies have still not been sufficiently validated, or have stagnated in terms of further development. Due to their high lethality, pelvic ring fractures are very significant with respect to stabilization and treatment.

For osteosynthesis of pelvic ring fractures, that is, for surgically attaching two bones, a simple plate implant is often implanted cranially, that is, on the head side, of the iliac crest. Ruptures of the symphysis (pelvic bone joint) of the human pelvis are also currently treated with simple plate implants, wherein the plate implant is attached from a cranial side to a first pubic bone and a second pubic bone of a patient to be treated, and the two hemispheres of pelvis are thus connected. Due to the great physiological stress, such simple plate implants often lead to implant failure, for example deformation or loosening of the implant.

It is further also known to use implants of more complex design than simple plates for treating pelvic ring fractures. RU 2 739 692 discloses a plate system for reconstruction of old pelvic ring fractures in male patients. The disclosed implant comprises a cranial plate connected to a second, U-shaped plate. The second plate is provided for placing on the pelvic bones to be connected from the outside. A disadvantage of the disclosed implant is that said implant is only intended for the treatment of male patients, and that the second plate must be attached to the pelvis from the outside. Significant patient impairment can occur when treating symphysis ruptures, due to the abdominal musculature positioned at the outside of the pelvis. The plate contacting the outside also impedes the view of the symphysis for a surgeon, whereby the risk of incorrect positioning of the implant rises. Only very experienced physicians can perform osteosynthesis by means of the proposed implant.

Furthermore, plate systems for treating pelvic ring fractures are known from CN 205126393 U and CN 204995566 U, but are not suitable for treating symphysis ruptures. The implant disclosed in CN 205126393 U is provided for osteosynthesis of fractures of the pelvic socket (acetabulum), while that disclosed in CN 204995566 U is provided for osteosynthesis of fractures of the sacroiliac joint. Typical problems with osteosynthesis of symphysis ruptures, such as correct adjustment of the symphysis spacing between the pubic bones, do not need to be considered for fractures of the acetabulum and the sacroiliac joint because the bones to be connected can simply be placed against each other. The implantation is not, or only slightly, hindered by the visual impeding of the surgeon.

The “PRO Pelvis and Acetabulum System” from the company Stryker is known. For treating fractures of the acetabulum, the system comprises “PRO quadrilateral surface plates” applied to the acetabulum from within the pelvic ring. Unlike the symphysis, for which the PRO Pelvis and Acetabulum System provides only simple plates, the pelvic socket is substantially easier to access and to view in the course of a surgical treatment, particularly because access typically is made through the patient's abdomen and thus allows an angled view of the pelvic socket.

The object of the present invention is to disclose a medical implant for osteosynthesis of symphysis rupture of the human pelvis comprising improved reliability with respect to implant failure relative to the prior art for enabling application with less patient distress and simpler implantation.

The object is achieved according to the invention by a medical implant of the type indicated above in a first consideration by a posterior plate implemented for being attached to the first pubic bone and the second pubic bone of the patient from a posterior side in order to connect the pubic bone, wherein the cranial plate and the posterior plate are connected to each other by means of at least one first connecting arm. The posterior side is the side of the pubic bones facing toward an inner side of the pelvic ring. The posterior side of the pubic bones face the sacrum of the patient in the region of the symphysis. The cranial side is the side facing toward the head of the patient. The implant according to the invention can accordingly be placed on the pubic bones of the patient under treatment from the inside and top in the course of osteosynthesis of a symphysis rupture. The inventors have discovered that it is possible to place a posterior plate from a posterior side of the pubic bones. Impairment of the abdominal musculature attached to the pubic bones from the outside can thereby be prevented. Common problems of implant failure, such as the fracture of a simple plate implant, are also overcome. This is achieved by more uniform distribution of the forces by the plates connected by means of the connecting arm. Forces and torques that arise can thus be optimally transferred to the screws in use and loosening of the implant is prevented. Furthermore, optimal alignment of the posterior plate and cranial plate to each other is achieved by the connecting arm. The medical implant can therefore be applied particularly simply and reliably, whereby less stressful treatment of the patient is made possible. The posterior plate and/or the cranial plate are preferably implemented for contacting the pubic bones of the patient substantially continuously (over the entire surface of the plates). It should be understood that the plates make continuous contact even when the symphysis is bridged. The posterior plate and/or the cranial plate are preferably adapted to a general geometry of the human pubic bones. In the course of osteosynthesis, a plate placed from the posterior side allows an unimpeded view of the symphysis by the surgeon. The implanting of the implant according to the invention is substantially facilitated.

The connecting arm connects the cranial plate and the posterior plate. A cross section of the connecting arm is preferably different from, particularly smaller than, a cross section of the posterior plate and/or of the cranial plate. The medical implant is preferably implemented having an L shape in a sagittal plane. The connecting arm is further preferably fully or partially flexible. It may be provided, however, that the connecting arm is fully or partially rigid in design. A connecting arm is rigid when said arm does not substantially deform during typical handling in the course of osteosynthesis. A flexible connecting arm is reversibly deformable, particularly already due to forces arising from the intrinsic weight of the implant. The connecting arm can be preferably curved, particularly in a circular arc, and/or polygonal in design. The medical implant can be used for both partial and complete symphysis ruptures.

According to a first preferred embodiment, the first connecting arm is an eccentric connecting arm relative to the posterior plate and the cranial plate, and is thus preferably disposed eccentrically along the longest extent of the cranial plate and/or posterior plate. For optimal force distribution, implants are typically aligned symmetrically to the symphysis in the course of osteosynthesis. An eccentric connecting arm ensures optimal accessibility to the symphysis, or accessibility is not impeded, because when the implant is largely symmetrically aligned, the connecting arm is disposed offset to the symphysis. Complications can be prevented.

The medical implant preferably comprises a second connecting arm, wherein the second connecting arm is particularly preferably an eccentric connecting arm relative to the posterior plate and cranial plate. The second connecting arm may preferably be implemented substantially identically to the first connecting arm. It may also be preferably provided, however, for example, that the second connecting arm has a different cross section than the first connecting arm. The first connecting arm and/or the second connecting arm are preferably connected substanstially perpendicular to the posterior plate and/or the cranial plate. Connecting arms connected perpendicular to the plates then extend substantially transverse to a pelvic ring direction. The pelvic ring direction is defined by the shape of the human pelvis in a transversal plane, that is, when viewed in the superior-inferior direction. The pelvic ring direction is approximately the circumferential direction of a circle or an ellipse. The first connecting arm and/or the second connecting arm may, however, also extend both transverse to and along the pelvic ring direction, for example at an angle to the pelvic ring direction.

In a preferred embodiment, the medical implant further comprises a positioning protrusion for at least partially engaging in the symphysis of the pelvis of the patient to be treated. The positioning protrusion can engage in the symphysis in the course of osteosynthesis and thus can be placed on a side of the pubic bone facing toward the symphysis in the pelvic ring direction. The positioning protrusion thus substantially facilitates the correct alignment of the medical implant on a first pubic bone. The implant can thus be attached to a first pubic bone in the correct alignment, before the two pubic bones or the two hip hemispheres of the patient are aligned to each other. The second hemisphere can then be precisely guided along the plate and attached. The hemispheres can be connected to each other horizontally and vertically by the two plates. By means of the implant according to the invention, it is no longer necessary to ensure the correct alignment of the pubic bones before the plates are attached to the two pubic bones. Alignment errors are effectively prevented and the patient is spared distress. The first connecting arm may preferably also be implemented as a positioning protrusion for at least partially engaging in the symphysis. The medical implant may also comprise a second positioning protrusion or more than two positioning protrusions.

The positioning protrusion is preferably disposed between the first connecting arm and the second connecting arm. At least one connecting arm is then preferably associated with each pubic bone of the patient, whereby the stability of the implant is improved. Furthermore, the placing of the positioning protrusion in the symphysis of the patient is not impeded by the connecting arms. The positioning protrusion is preferably disposed centered between the connecting arms, thus has substantially the same spacing from the first connecting arm and the second connecting arm. The connecting arm is preferably implemented integrally with the posterior plate and/or the cranial plate.

In a preferred embodiment, the positioning protrusion is disposed centered on the implant, preferably in a plane of symmetry of the implant. The positioning protrusion is disposed centered as seen in the pelvic ring direction. The positioning protrusion is preferably disposed centered between a first end of the medical implant farthest away from the symphysis in the pelvic ring direction when the positioning protrusion is positioned in the symphysis, and an opposite, second end of the medical implant farthest away from the symphysis in the pelvic ring direction when the positioning protrusion is positioned in the symphysis. The positioning protrusion is preferably disposed in a plane of symmetry of the implant perpendicular to the pelvic ring direction.

The positioning protrusion preferably comprises a blunt end face. The end face is preferably rounded off in design and particularly preferably comprises no edges. A blunt end face prevents injury to the symphysis and/or to the sides of the pubic bones facing towards the symphysis. The end face of the positioning protrusion is preferably dome-shaped in design. It may also be provided, however, that the end face is flat and comprises rounded transitions to side walls of the protrusion. In a preferred refinement, the positioning protrusion is a ridge elongated transverse to a protrusion height of the protrusion. An elongated ridge provides a particularly large contact surface and thus allows secure positioning. The protrusion height is determined in the direction in which the positioning protrusion engages in the symphysis when the implant is used as intended. If the positioning protrusion extends from the cranial plate, then the protrusion height is determined in the superior-inferior direction. If, however, the positioning protrusion extends from the posterior plate, then the protrusion height is determined in the posterior-anterior direction.

In a preferred embodiment, the positioning protrusion extends substantially perpendicular from a contact surface on the cranial plate and/or the posterior plate of the medical implant. Slipping of the positioning protrusion can thus be effectively prevented. It may also be provided, however, that the positioning protrusion extends at an angle from the contact surface. For example, the positioning protrusion may be implemented having a wedge shape. A wedge-shaped positioning protrusion facilitates inserting into the symphysis.

The positioning protrusion is preferably provided on the posterior plate. By disposing the positioning protrusion on the posterior plate, correct seating of the implant can be appraised well in the course of the osteosynthesis. The risk of incorrect position and/or of complications is reduced. The positioning protrusion may also, however, be provided on the cranial plate. For example, the positioning protrusion extends completely transverse to the protrusion height from an inferior side of the posterior plate up to a superior side of the posterior plate. A particularly large contact surface is thereby achieved.

The positioning protrusion preferably comprises a protrusion height in a range from 0.5 mm to 20 mm, preferably 0.5 m to 15 mm, preferably 0.5 mm to 12 mm, preferably 1 mm to 12 mm, preferably 1 mm to 10 mm, preferably 1 mm to 8 mm, preferably 2 mm to 8 mm, preferably 2 mm to 6 mm, preferably 2 mm to 5 mm, particularly preferably 3 mm to 5 mm. The boundary values of the ranges are also preferred. A protrusion height in the claimed range enables secure contact and simultaneously does not hinder the healing process of the symphysis.

In a further preferred embodiment, the posterior plate comprises first attaching holes provided for receiving first connecting means, wherein the cranial plate comprises second attaching holes provided for receiving second connecting means, and wherein the first attaching holes or the second attaching holes are implemented as oval holes. The first attaching holes are preferably implemented as oval holes. Oval holes have a greater extent in a first direction transverse to a pass-through direction in which connecting means extend through holes than in a second direction. Oval holes have a cross section different than a circular shape. Thus, oval holes can be implemented as ovals or ellipses, for example. Oval holes may, however, preferably also be elongated holes having parallel sides, or rectangular transverse to the pass-through direction. It should be understood that oval holes are not limited to an oval cross section. Oval holes allow drilling with a major axis of the drill tilted relative to the pass-through direction of the oval hole. In the course of osteosynthesis, attachment holes must often be drilled in a bone of the patient to be treated, in which the attaching means are anchored. Accessibility in the course of an operation is limited. Oval holes make it possible that a drill used for drilling need not be held perpendicular to the bone, and thus simplify medical treatment of the patient. Injuries to the surrounding tissue are minimized, defects are prevented, and the patient is protected. It may also be provided, alternatively, that both the first attachment holes and the second attachment holes are implemented as oval holes.

A longitudinal axis of the oval holes is preferably substantially oriented along a pelvic ring direction. The longitudinal axis indicates the greatest opening size of the oval holes transverse to the pass-through direction. The inventor has discovered that drilling can be further facilitated by orienting the longitudinal axis of the oval holes in the pelvic ring direction.

In a preferred refinement, the connecting arm comprises a lower rigidity than the posterior plate and the cranial plate, so that the alignment of the posterior plate can be adjusted relative to the cranial plate by plastically deforming the connecting arm. If the medical implant comprises a plurality of connecting arms, then an overall rigidity of the connecting arms is preferably less than the rigidity of the posterior plate and the cranial plate. Due to the lower rigidity, the connecting arm can be deformed, while the posterior plate and the cranial plate do not or do not substantially deform. The medical implant can be adapted to the specific characteristics of the patient to be treated by means of plastic deformation. Thus, an angle enclosed by the plates may preferably be varied. The relevant rigidity is preferably a rigidity with respect to deforming in a plane transverse to the pelvic ring direction (e.g., a sagittal plane running through the symphysis). The connecting arm preferably has a length of about 25 mm and preferably a width of about 10 mm.

The cranial plate is preferably convex in design in a transversal plane and/or the posterior plate is convex in design in the transversal plane. A convex plate according to the invention is curved toward the abdomen of the patient or is curved outwardly. A convex posterior plate is particularly well adapted to the natural shape of the pelvic ring. Due to a convex cranial plate, a particularly large contact surface is achieved. Convex plates further prevent the implant from protruding after implanting and thus prevent injuries to the surrounding tissue.

In one embodiment, a first implant segment for attaching to the first pubic bone and a second implant segment for attaching to the second pubic bone are implemented substantially mirror-symmetrical to each other. The first implant segment comprises such segments of the posterior plate and the cranial plate provided for attaching to the first pubic bone and the second implant segment analogously comprises the segments of the posterior plate and the cranial plate provided for attaching to the second pubic bone. A mirror-symmetrical design allows symmetrical force distribution and thus prevents loosening of and/or damage to the implant. It should be understood that implant segments between the first and the second implant segment need not be mirror-symmetrical in design, but may be the same.

The cranial plate, the posterior plate, and/or the connecting arm or the entire implant are preferably made of steel, titanium, a powder-coated metal, and/or a medical plastic, preferably PEEK. The cranial pate, the posterior plate, the connecting arm, and/or the positioning protrusion may also preferably comprise surface structuring. The medical implant is particularly preferably produced by means of 3D printing (additive manufacturing).

A length of the posterior plate and a length of the cranial plate are preferably substantially identical. The length of the plates is determined in the pelvic ring direction. Designing the plates to have the same length may achieve introducing forces optimally and may prevent implant failure.

In a preferred embodiment, the at least one connecting arm is disposed spaced apart in a pelvic ring direction from ends of the posterior plate and/or ends of the cranial plate. The Contractor is thus not disposed on the ends of the posterior plate lying in the pelvic ring direction of the plates, and/or of the cranial plate. If the implant comprises a plurality of connecting arms, then preferably all connecting arms are implemented spaced apart from the ends of the plates.

According to a second consideration of the invention, the object stated above is achieved by a method for osteosynthesis of an artificial symphysis rupture on a training body for training purposes by means of a medical implant, preferably by means of a medical implant according to the above described first consideration of the invention, comprising the steps: placing a cranial plate of the medical implant on a first pubic bone of the training body from a cranial side; placing a posterior plate of the medical implant on a first pubic bone of the training body from a posterior side; attaching the cranial plate to the first pubic bone; attaching the posterior plate to the first pubic bone; adjusting a symphysis spacing between the first pubic bone and the second pubic bone; attaching the cranial plate to the second pubic bone and attaching the posterior plate to the second pubic bone after adjusting the symphysis spacing. The training body is not a living human or animal body. The method according to the invention allows effective training of osteosynthesis of a symphysis rupture. The steps of placing the cranial plate on the first pubic bone and placing the posterior plate on the first pubic bone can be performed in any arbitrary sequence, preferably simultaneously. In an analogous manner, the attaching of the cranial plate and the attaching of the posterior plate to the first pubic bone can take place in any arbitrary sequence, preferably simultaneously. Adjusting the symphysis spacing preferably takes place after attaching the posterior plate and/or the cranial plate to the first pubic bone of the training body.

In a first preferred refinement, the method further comprises: placing a positioning protrusion of the medical implant on the first pubic bone from a side facing toward the symphysis of the training body, wherein the placing of the positioning protrusion is performed before attaching the cranial plate and/or the posterior plate on the first pubic bone. Preferably, for the method according to the invention, the medical implant is first attached to the first pubic bone, wherein correct position of the implant is ensured by the positioning protrusion placed on the first pubic bone. The implant can thus first be positioned correctly on and attached to the first pubic bone before the symphysis spacing is adjusted and the implant, that is, the posterior plate and the cranial plate, are also attached to the second pubic bone. The method is significantly easier to learn in comparison with typical training methods.

Embodiments of the invention are described below using the drawings. Said drawings are not necessarily intended to depict the embodiments to scale, but rather the drawings are schematic and/or slightly distorted as needed for better explanation. With respect to additions to the teachings directly evident from the drawings, reference is made to the relevant prior art. It must thereby be considered that multiple modifications and changes relating to the shape and the detail of an embodiment can be made without deviating from the general idea of the invention. The features of the invention disclosed in the description, in the drawings, and in the claims may be essential to the refinement of the invention both individually and in any arbitrary combination. All combinations of two or more of the features disclosed in the description, the drawings, and/or the claims also fall within the scope of the invention. The general idea of the invention is not limited to the exact shape or the detail of the preferred embodiments shown and described below, nor limited to one subject-matter that would be limited in comparison with the subject-matter claimed in the claims. Where dimension ranges are given, values within the indicated limits should also be disclosed as limit values and should be able to be applied and claimed arbitrarily. For simplicity, the same reference numerals are used below for identical or similar parts, or for parts having identical or similar functions.

Further advantages, features, and details of the invention arise from the following description of the preferred embodiment and from the drawings; said drawings showing in:

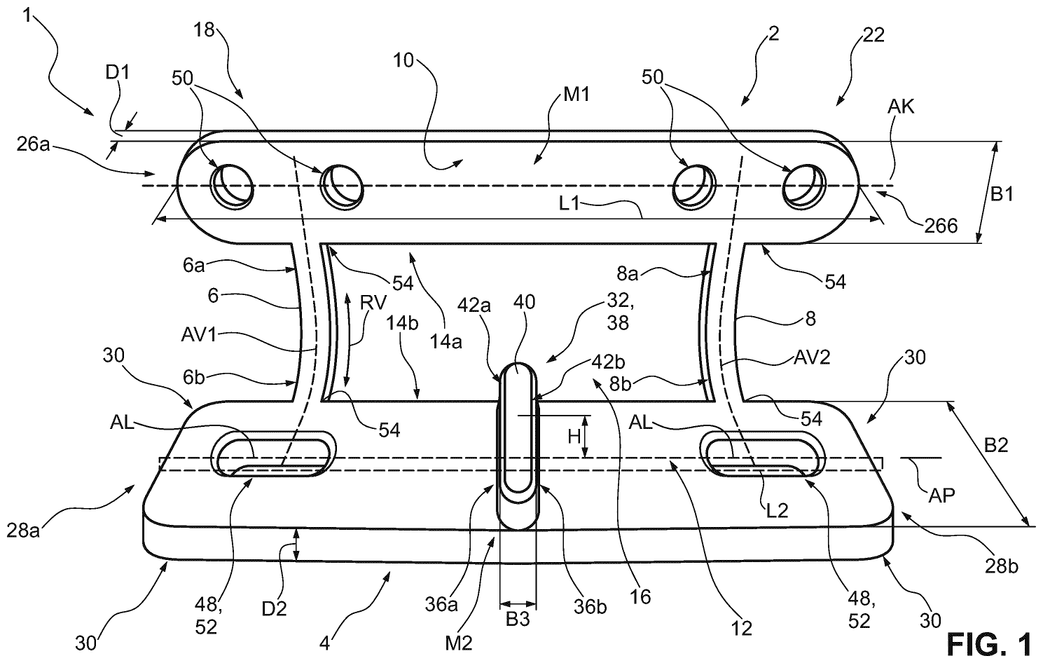

FIG. 1 a medical implant in a first embodiment;

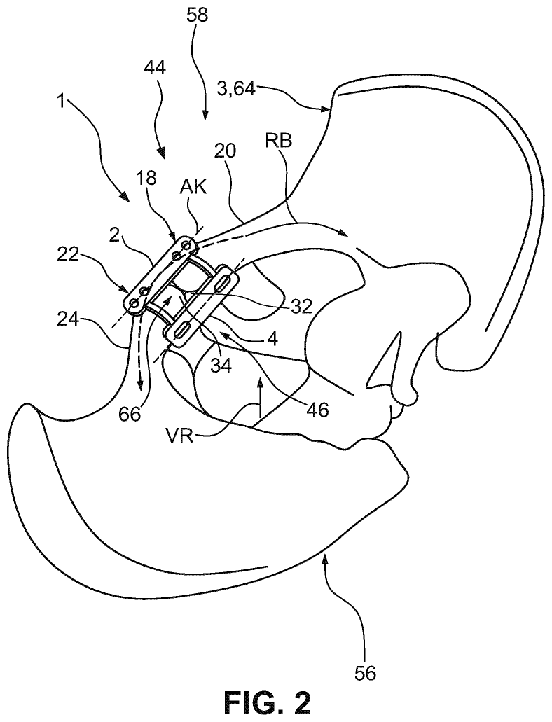

FIG. 2 the medical implant according to the first embodiment placed on a model of a human hip bone;

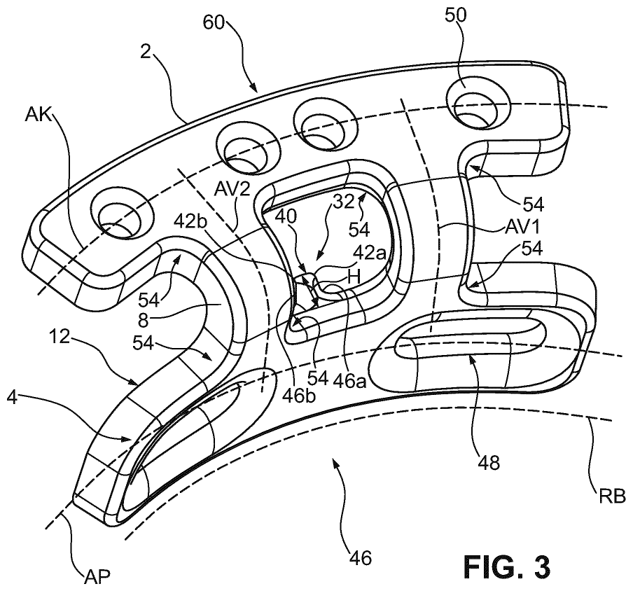

FIG. 3 a medical implant in a second embodiment; and

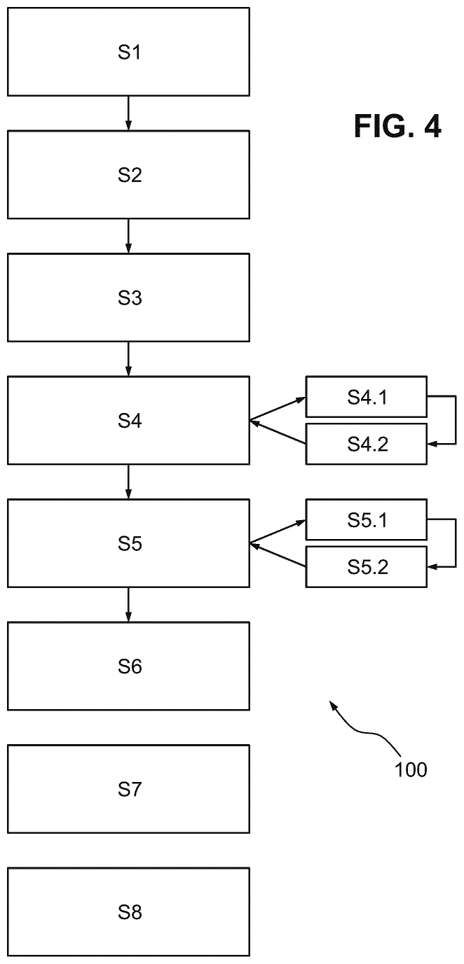

FIG. 4 an illustrated depiction of the method according to the invention.

FIG. 1 shows a medical implant 1 for osteosynthesis of symphysis ruptures on the human pelvis, comprising a cranial plate 2 and a posterior plate 4. The cranial plate 2 and the posterior plate 4 are connected to each other by a first connecting arm 6 and a second connecting arm 8. For placing on a first pubic bone 20 and a second pubic bone 24 of a patient to be treated (not shown), the cranial plate 2 comprises a cranial contact surface 10. In an analogous manner, the posterior plate 4 comprises a posterior contact surface 12 for placing on the pubic bones 20, 24.

The connecting arms 6, 8 are implemented at angles, so that the cranial contact surface 10 of the cranial plate 2 and the posterior contact surface 12 of the posterior plate 4 are disposed at an angle to each other. In the present embodiment example, the posterior contact surface 12 and the cranial contact surface 10 enclose an angle of approximately 90°. An angled arrangement of the contact surfaces 10, 12 may also, however, be achieved by connecting arms 6, 8 having a curved shape, curved in a plurality of axes, or having a plurality of bends. The first connecting arm 6 extends from a lateral surface 14a of the cranial plate 2 facing toward the posterior plate 4 to a lateral surface 14b of the posterior plate 4 facing toward the cranial plate 2. The second connecting arm 8 extends in an analogous manner between the lateral surface 14a of the cranial plate 2 and the lateral surface 14b of the posterior plate 12. The first connecting arm 6 is implemented at an angle, so that a first arm segment 6a is implemented substantially perpendicular to a second arm segment 6b, wherein the first arm segment 6a is connected to the cranial plate 10 and the second arm segment 6b is connected to the posterior plate 12. The segment 6a, 6b transition into each other perpendicularly. In an analogous manner, a first arm segment 8a and a second arm segment 8b of the second connecting arm 8 are connected to the cranial plate 10 and the posterior plate 12.

The first connecting arm 6 and the second connecting arm 8 are implemented substantially symmetrically here. The connecting arms 6, 8 comprise a substantially rectangular cross section having rounded corners transverse to a connecting direction RV. The connecting direction is curved or bent due to the perpendicular arrangement of the arm segments 6a, 6b, 8a, 8b and runs along the connecting arms 6, 8. The connecting arms 6, 8 extend laterally from the cranial plate 10 and the posterior plate 12, wherein a longitudinal axis AV1 of the first connecting arm 6 is perpendicular to a longitudinal axis AK of the cranial plate 2. The longitudinal axis AV1 of the first connecting arm 6 is also substantially perpendicular to a longitudinal axis AP of the posterior plate 4. In an analogous manner, a longitudinal axis AV2 of the second connecting arm 8 is also substantially perpendicular to the longitudinal axis AK of the cranial plate 2 and to the longitudinal axis AP of the posterior plate 4. The longitudinal axes AV1, AV2, AK, AP indicate the greatest geometric extent of each of the connecting arms 6, 8, the cranial plate 2, and the posterior plate 4.

The connecting arms 6, 8 are implemented as eccentric connecting arms 6, 8. Thus the first connecting arm 6 is connected to the cranial plate 2 spaced apart from a center M1 of the cranial plate 2, and connected to the posterior plate 4 spaced apart from a center M2 of the posterior plate 4. In an analogous manner, the second connecting arm 8 is also connected to the cranial plate 2 and to the posterior plate 4 spaced apart from the first center M1 of the cranial plate 2 and from the second center M2 of the posterior plate 4. A viewing window 16 is thus formed between the connecting arms 6, 8, facilitating the aligning of the medical implant 1 in the course of osteosynthesis of a symphysis rupture. A symphysis 34 of the patient to be treated is not obscured by the connecting arms 6, 8. In the present embodiment example, the medical implant 1 is mirror-symmetrical in design, wherein the plane of symmetry, not shown, extends through the center M1 of the cranial plate 2 and the center M2 of the posterior plate 4. A first implant segment 18 for placing on a first pubic bone 20 (FIG. 2) and a second implant segment 20 for placing on a second pubic bone 24 are substantially identical in design. If, however, an osteosynthesis of multiple fractures (comprising a symphysis rupture) is to be treated, for example, then the medical implant 1 can be asymmetrical in design. For example, the first implant segment 18 can be implemented longer in the pelvic ring direction RB than the second implant segment 22, in order to thus enable connecting fragments of the first pubic bone 20.

The cranial plate 2 is implemented as a flat plate, wherein the longitudinal axis AK of the cranial plate 2 is straight. The longitudinal axis AK defines a longitudinal direction of the cranial plate 2. Two ends 26a, 26b of the cranial plate 2, bounding the cranial plate 2 in said longitudinal direction, are rounded off. The cranial contact surface 10 of the cranial plate 2 is substantially rectangular and has two semicircular segments at the ends 26a and 26b.

The posterior plate 4 also comprises a straight longitudinal axis AP defining a longitudinal direction of the posterior plate 4. The contact surface 12 of the posterior plate 4 is substantially rectangular, wherein the ends 28a, 28b bounding the posterior plate 4 in the longitudinal direction comprise rounded corners 30. Between the corners 30, the posterior contact surface 12 is bounded by straight lines. Unlike the cranial plate 2, the ends 28a, 28b of the posterior plate 4 in the present embodiment example are thus not semicircular in design.

The ends 26a, 26b of the cranial plate 2 protrude from the connecting arms 6, 8 in the longitudinal direction of the cranial plate 2. Because the longitudinal axis AK of the cranial plate 2 extends substantially along a pelvic ring direction RB, the ends 26a, 26b also protrude from the connecting arms 6, 8 in the pelvic ring direction RB. The connecting arms 6, 8 are not connected to the ends 26a, 26b, but rather flow into the cranial plate 2 between the center M1 and the ends 26a, 26b.

The ends 28a, 28b of the posterior plate 4 protrude from the connecting arms 6, 8 in the longitudinal direction of the posterior plate 4 in an analogous manner, so that the connecting arms 6, 8 flow into the posterior plate 4 between the center M2 and the ends 28a, 28b of the posterior plate 4. The longitudinal axis AP of the posterior plate 4 also extends substantially in the pelvic ring direction RB, so that the ends 28a, 28b of the posterior plate 28a, 28b also protrude from the connecting arms 6, 8 in the pelvic ring direction RB.

It may also be provided, however, that connecting arms 6, 8 flow into the ends 26a, 26b of the cranial plate 2 and/or the ends 28a, 28b of the posterior plate 4. Furthermore, the connecting arms 6, 8 may enclose an angle other than 900 with the longitudinal axis AK of the cranial plate 2 and/or the longitudinal axis AP of the posterior plate 4. For example, the connecting arms 6, 8 may also be disposed in a V shape.

A length L1 of the cranial plate 2 is determined along the longitudinal axis AK and a length L2 of the posterior plate 4 is determined along the longitudinal axis AP. The length L1 of the cranial plate 2 is substantially identical to the length L2 of the posterior plate 4. A width B1 of the cranial plate 2 is determined transverse to the longitudinal axis AK. In an analogous manner, a width B2 of the posterior plate 4 is determined transverse to the longitudinal axis AP of the posterior plate 4. In the present embodiment example, the width B1 of the cranial plate 2 is less than the width B2 of the posterior plate 4. Thus the cranial contact surface 10 of the cranial plate 2 is smaller than the posterior contact surface 12 of the posterior plate 4. A thickness D1 of the cranial plate 2 determined transverse to the cranial contact surface 10 and transverse to the longitudinal axis AK of the cranial plate 2 is substantially identical to a thickness D2 of the posterior plate 4 as measured transverse to the posterior contact surface 12 and transverse to the longitudinal axis AP of the posterior plate 4. It may also be provided, however, that the thicknesses D1, D2 of the cranial plate 2 and the posterior plate 4 are different. Thus, for example, a rigidity of the plates 2, 4 with respect to deforming may be adapted to stresses occurring after implanting, in order to thus prevent failure of the implant.

A positioning protrusion 32 extending from the posterior plate 32 transverse to the pelvic ring direction RB is implemented on the posterior plate 4. The positioning protrusion 32 is implemented for engaging in the symphysis 34 (FIG. 2). Here the positioning protrusion 32 extends substantially perpendicular from the posterior plate 4, so that side walls 46a, 46b of the positioning protrusion 32 are disposed substantially perpendicular to the posterior contact surface 12. The positioning protrusion 32 is implemented as an elongated ridge 38 extending over the entire width B2 of the posterior plate 4. Alternatively, however, the positioning protrusion may also be preferably implemented as a cylinder, cone, or wedge extending from a contact surface 10, 12.

The positioning protrusion 32 is disposed centered on the posterior plate 4 and extends from the second center M2 thereof. An end face 40 of the positioning protrusion 32 is blunt, that is, has no sharp edges. Transitions 42a, 42b of the end face 40 into the side walls 46a, 46b of the positioning protrusion 32 are rounded off, so that a risk of damaging surrounding tissue when placing the positioning protrusion into the symphysis 34 is minimized. The end face 40 here is substantially parallel to the posterior contact surface 12. It may also be provided, however, that the end face 40 forms a (preferably rounded) point or is dome-shaped or arch-shaped in design. In addition or alternatively, the positioning protrusion 32 may also be provided on the cranial plate 2.

A protrusion height H of the positioning protrusion 32 is determined transverse to the contact surface 12. If the cranial plate 2 contacts the first pubic bone 20 and the second pubic bone 24 from a cranial side 44, and if the posterior plate 4 contacts the first and second pubic bones 20, 24 from a posterior side 46, then the protrusion height H is a measure of how far the positioning protrusion 32 engages into the symphysis 34. Preferably, a protrusion width B2 of the positioning protrusion 32 as determined in the pelvic ring direction RB is adapted to the width of a human symphysis. In the course of osteosynthesis of a symphysis rupture, the correct positioning of the medical implant 1 and adjusting of the symphysis spacing between the first pubic bone 20 and the second pubic bone 24 is substantially facilitated. Thus the first implant segment 18 may first be placed on the first pubic bone 20, wherein the positioning protrusion 32 engaging in the symphysis 34 ensures an optimal position of the medical implant 1 in the pelvic ring direction RB. Subsequently, the first implant segment 18 may be attached to the first pubic bone 20. The medical implant 1 is then fixedly connected to the first pubic bone 20. Subsequently, the symphysis gap between the first pubic bone 20 and the second pubic bone 24 may by adjusted, in that the second pubic bone 24 is brought into contact with the positioning protrusion 32 on a side opposite the first pubic bone 20. The second implant segment 22 is subsequently connected to the second pubic bone 24 for connecting the two pubic bones 20, 24.

The posterior plate 4 comprises first connecting holes 48 for connecting the medical implant 1 to the first pubic bone 20 and the second pubic bone 24. The cranial plate 2 further comprises two connecting holes 50. The course of osteosynthesis, first connecting means (not shown) may be inserted through the first connecting holes 48 of the posterior plate 4 and second connecting means (not shown) may be inserted through the second connecting holes 50 of the cranial plate 2. Such connecting means are typically medical screws, nails, and/or pins. A head segment of the connecting means then contacts a side of the plates 2, 4 opposite the contact surfaces 10, 12 and clamps the posterior plate 4 or the cranial plate 2 against the first pubic bone 18 or the second pubic bone 24.

The posterior plate 4 in the embodiments shown comprises two first connecting holes 48 implemented as oval holes 52. It should be understood that the posterior plate 4 may also comprise only one first connecting hole 48 or more than two first connecting holes 48. Furthermore, not all first connecting holes 48 must be implemented as oval holes 52. Longitudinal axes AL of the oval holes 52 contact the longitudinal axis AP of the posterior plate 4. The longitudinal axis AL of the oval holes 52 indicates a maximum clearance width of the oval holes 52 transverse to such direction in which the attaching means are inserted through the oval holes. Here the oval holes 52 are formed from two semicircular segment connected by means of segments parallel to the longitudinal axis AL of the oval holes 52. Connecting holes 48 having such a cross-sectional geometry are also often referred to as elongated holes. The oval holes 52 are on the longitudinal axis AP of the posterior plate 4 extending substantially along the pelvic ring direction RB, so that the longitudinal axis AL of the oval holes 52 also extends substantially along the pelvic ring direction RB. The oval holes 52 facilitate drilling of attaching holes (not shown) in the first pubic bone 20 and the second pubic bone 24 that must be drilled in the course of osteosynthesis in order to enable attaching the implant 1. A drill, not shown, need not be guided perpendicular to the posterior side 46 when drilling, but rather may also be applied at an angle.

The cranial plate 2 comprises a total of four second attaching holes 50, implemented here as round holes. Transverse to the direction in which the attaching means are inserted through the holes 50 (the pass-through direction), the attaching holes 50 have a circular cross section. It may also be provided, however, that some or all of the second connecting holes 50 are implemented as oval holes 52. In the course of osteosynthesis, accessibility from the cranial side 44 can typically be better ensured than accessibility of the posterior side 46, so that at least the posterior plate 4 preferably comprises oval holes 52. Round holes, in contrast, advantageously prevent the attaching means from slipping. The first attaching holes 48 and/or the second attaching holes 50 may advantageously be conical in design in the pass-through direction.

The first attaching holes 48 and/or the second attaching holes 50 are preferably disposed in the region of the connecting arms 6, 8. The longitudinal axis AV1 of the first connecting arm 6 thus preferably extends between the attaching holes 50 of the first implant segment 18 on the cranial plate 2. In an analogous manner, the second longitudinal axis AV2 of the second connecting arm 8 extends between the attaching holes 50 of the second implant segment 20 on the cranial plate 2. On the posterior plate 4, the first longitudinal axis AV1 of the first connecting arm 6 and/or the second longitudinal axis AV2 of the second connecting arm 8 extends through the elongated holes 52 on the first implant segment 18 or on the second implant segment 22.

The first connecting holes 48 and/or the second connecting holes 50 are preferably disposed centered on the cranial plate 2 or the posterior plate 4 in a width direction running parallel to the first and second widths B1, B2.

A cross section of the connecting arms 6, 8 is selected so that the connecting arms 6, 8 have a lower rigidity than the cranial plate 2 an the posterior plate 4. The connecting arms 6, 8 may thereby be plastically deformed in order to modify an alignment of the cranial contact surface 10 to the posterior contact surface 12. Thus the angled arrangement of the cranial contact surface 10 to the posterior contact surface 12 may be modified by bending up the connecting arms 6, 8. After bending up, the cranial contact surface 10 and the posterior contact surface 12 enclose an angle of greater than 90°. In an analogous manner, the medical implant 1 may also be bent together, so that the cranial contact surface 10 and the posterior contact surface 12 enclose an angle of less than 90°. It should be understood that the contact surfaces 10, 12 may also comprise an angle other than 90° prior to bending up or bending together. For plastic deforming of one or both connecting arms 6, 8, the shape of the longitudinal axis AV1 of the first connecting arm 6 and/or of the second connecting axis AV2 of the second connecting arm 8 changes. The cranial plate 2 and the posterior plate 4, the rigidity of which is determined substantially by the width B1, B2 and the thicknesses D1, D2 for the integral embodiment shown, are not deformed and remain flat. The plastically deformable connecting arms 6, 8 thus enable adapting the medical implant 1 to the shape of the first pubic bone 20 and the second pubic bone 24. The connecting arms 6, 8 can preferably be deformed without tools.

When plastically deformed, the longitudinal axes AV1, AV2 of the connecting arms 6, 8 are curved. It may also be provided, however, that transition regions 54 of the connecting arms 6 to the plates 2, 4 are deformed, while the shape of het connecting arm 6, 8 remains substantially constant. A cross-sectional area (not shown) of the connecting arms 6, 8 transverse to the longitudinal axes AV1, AV2 thereof is selected accordingly, so that the rigidity of the connecting arms 6, 8 is less than the rigidity of the plates 2, 4. The cross-sectional geometry of the connecting arms 6, 8 further influences the rigidity of the connecting arms, wherein round, oval, rectangular, square, I-shaped, T-shaped, L-shaped, and/or annular cross-sectional shapes of the connecting arms are preferred. It may also be provided, however, that at least one of the connecting arms 6, 8 comprises a deforming segment, not shown, made of a more easily deformable material than the plates 2, 4. For example, the connecting arms 6, 8 may be made of titanium in segments, while the posterior plate 4 and the cranial plate 8 are made of steel.

FIG. 2 clarifies the arrangement of the medical implant 1 in the course of osteosynthesis of a symphysis rupture on a human pelvis 3, wherein instead of a real human pelvis 3, a training body 64 is shown, accurately simulating the details of the natural shape of a human pelvis. The cranial plate 2 is placed on the first pubic bone 18 and the second pubic bone 24 from the cranial side 44. The cranial plate 2 thus bridges the symphysis 34 from the cranial side 44. Forces applied to the pubic bones 20, 24 can be transmitted by the cranial plate 2 from the first pubic bone 20 to the second pubic bone 24, and vice versa. The posterior plate 4 is place on the pubic bones 20, 24 from the posterior side 46 and also bridges the symphysis 34. The positioning protrusion 32 engages in the symphysis 34 when the medical implant 1 is correctly positioned on the pelvis 3.

In the first embodiment shown in FIG. 2, the cranial plate 2 and the posterior plate 4 are straight and flat plates. Nevertheless, the posterior plate 4 and the cranial plate 4 extend substantially along the pelvic ring direction RB. The positioning protrusion 32 implemented as a ridge 38 extends from an inferior side 56 to a superior side 58 of the pelvis in a vertical direction VR. Perpendicular to the posterior plate 4, the positioning protrusion 32 does not extend entirely through the symphysis 34 from the posterior contact surface 12.

FIG. 3 clarifies a second embodiment of the medical implant 1. In the second embodiment, the shape of the medical implant 1 is further adapted to a natural shape of the human pelvis 3. Unlike in FIG. 1, which shows a side of the medical implant 1 facing toward the pubic bones 20, 24, FIG. 3 clarifies a view of a side of the medical implant 1 facing away from the pubic bones 20, 24.

The second embodiment according to FIG. 3 is implemented substantially analogous to the first embodiment according to FIGS. 1 and 2, wherein identical segments and components are labeled with identical reference numerals. In contrast to the first embodiment, the cranial plate 2 and the posterior plate 4 according to the second embodiment example are implemented not as straight plates, but curved. The cranial plate 2 is also curved and the cranial contact surface 10 is a flat surface. The longitudinal axis AK of the cranial plate 2 is convexly curved in a transversal plane substantially parallel to the plane of the image in FIG. 3, so that a bulge 60 of the cranial plate 2 faces away from the posterior side 46. A protrusion of the cranial plate 2 past the pubic bones 20, 24 after osteosynthesis is complete is thus minimized while simultaneously maximizing the contact surface.

The posterior plate 4 is also curved in design. The longitudinal axis AP of the posterior plate 4 is convex. A bulge 62 of the posterior plate 4 faces away from the posterior side 46, that is, in the same direction as the bulge 60 of the cranial plate 2. The posterior contact surface 12, in contrast to the first embodiment example, is not flat, but rather curved in an arch shape. Her the posterior plate 4 is curved in only one axis (only the longitudinal axis AP of the posterior plate 4 is curved). It may also be provided, however, that the posterior plate is curved in a plurality of axes. By means of the curvature, the posterior contact surface 12 is optimally adapted to the shape of the pubic bones 20, 24 of the pelvis 3. Alternatively to the embodiment shown, it may be provided that the posterior contact surface 12 is curved, while a surface of the posterior plate 12 opposite the posterior contact surface 12 is flat.

Furthermore, the connecting arms 6, 8 of the second embodiment of the medical implant 1 have a larger cross section in comparison with the first embodiment. A resistance of the connecting arms 6, 8 to deforming is increased, so that forces applied to the plates 2, 4 can be better transmitted between the plates 2, 4. However, a greater force must be applied for adapting the medical implant 1 to the pelvis 3. The transitions 54 implemented between the connecting arms 6, 8 and the plates 2, 4 are rounded, whereby the strength of the medical implant 1 is improved and impairment of surrounding tissue can be prevented.

FIG. 4 illustrates a method 100 for osteosynthesis of a symphysis rupture on a training body 64 for training purposes by means of a medical implant 1. In a first step S1, the cranial plate 2 of the medical implant 1 is placed on the first pubic bone 20 of the training body 64. The placing takes place from the cranial side 44.

In a second step S2, the posterior plate 4 of the medical implant 1 is placed on the first pubic bone 20 of the training body 64, wherein the placing takes place from the posterior side 46. It should be understood that the steps S1 and S2 may be performed simultaneously or in reversed sequence.

In a third step S3, the positioning protrusion 32 of the medical implant 1 is placed on the first pubic bone 20 from a side facing toward the symphysis 34 of the training body 64. Optimal positioning of the first implant segment 18 to the first pubic bone 20 is thereby ensured.

Subsequently, in a fourth step S4, the cranial plate 2 is attached to the first pubic bone 20. For this purpose, in a first partial step S4.1, attaching holes (not shown) may first be drilled in the first pubic bone 20 through the second connecting holes 50. Subsequently, in a second partial step S4.2, connecting means are inserted through the second connecting holes 50 and inserted into the previously drilled attaching holes, whereby the cranial plate 2 is attached to the first pubic bone 18.

In a fifth step S5, the posterior plate 4 is attached to the first pubic bone 20. Analogous to the fourth step S4, in a first partial step S5.1 of the fifth step S5, attaching holes (not shown) may also first be drilled in the first pubic bone 20 through the first connecting holes 48. In a second partial step S5.2, connecting means (not shown) may then be inserted through the first connecting holes 48 and inserted into the attaching holes in order to attach the posterior plate 4 to the first pubic bone 20. The first partial step S5.1 may optionally be eliminated and the connecting means, such as screws, may be inserted directly. The steps S4 and S5 may be performed in any arbitrary sequence, for example in parallel or in reverse sequence. For example, the partial steps S4.1 and S5.1 may be performed first, before the posterior plate 4 and the cranial plate 2 are attached to the first pubic bone 20 by means of the attaching means (S5.2, S4.2). Alternatively, for example, the posterior plate 4 may be attached to the first pubic bone 20 before the cranial plate 2.

In a subsequent sixth step S6, a symphysis spacing as seen in the pelvic ring direction RB between the first pubic bone 20 and the second pubic bone 24 is adjusted. For this purpose, the second pubic bone 24 is preferably displaced toward the first pubic bone 20 until the first pubic bone 24 contacts the positioning protrusion 32. The positioning protrusion 32 and the width B3 thereof define the symphysis spacing of the symphysis 34. The aligning of the pubic bones 20, 24 of the training body 64 to each other is facilitated.

In a seventh partial step S7, the cranial plate 2 is connected to the second pubic bone 24, wherein the second pubic bone 20 is first placed on the cranial plate 2. The seventh step S7 may comprise partial steps, analogous to the fourth step S4.

In an eighth step S8, the second pubic bone 24 is attached to the posterior plate 4, wherein the second pubic bone 24 is first placed on the posterior plate 4. The eighth step S8 may comprise partial steps, analogous to the fifth step S5. Furthermore, the steps S7 and S8 may be performed in any arbitrary sequence.

1. A medical implant for osteosynthesis of symphysis ruptures on the human pelvis, comprising
a cranial plate implemented for being attached from a cranial side to a first pubic bone and a second pubic bone of a patient to be treated in order to connect the pubic bones,
a posterior plate implemented for being attached from a posterior side to the first pubic bone and the second pubic bone of the patient in order to connect the pubic bones,
wherein the cranial plate and the posterior plate are connected to each other by means of at least one first connecting arm.

2. The medical implant according to claim 1, wherein the first connecting arm is an eccentric connecting arm relative to the posterior plate and the cranial plate.

3. The medical implant according to claim 1, further comprising a second connecting arm, wherein the second connecting arm is preferably an eccentric connecting arm relative to the posterior plate and the cranial plate.

4. The medical implant according to claim 1, further comprising a positioning protrusion for at least partially engaging in the symphysis of the pelvis of the patient to be treated.

5. The medical implant according to claim 3, wherein the positioning protrusion (32) is disposed between the first connecting arm and the second connecting arm.

6. The medical implant according to claim 4, wherein the positioning protrusion is disposed centered on the implant, preferably in a plane of symmetry of the implant.

7. The medical implant according to claim 4, wherein the positioning protrusion comprises a blunt end face.

8. The medical implant according to claim 4, wherein the positioning protrusion is an elongated ridge transverse to a protrusion height of the positioning protrusion.

9. The medical implant according to claim 4, wherein the positioning protrusion extends substantially perpendicular from a contact surface on the cranial plate and/or the posterior plate of the medical implant.

10. The medical implant according to claim 4, wherein the positioning protrusion is provided on the posterior plate.

11. The medical implant according to claim 4, wherein the positioning protrusion comprises a protrusion height in a range from 1 mm to 15 mm, preferably 2 mm to 5 mm.

12. The medical implant according to claim 1, wherein the posterior plate comprises first attaching holes provided for receiving first connecting means, wherein the cranial plate comprises second attaching holes provided for receiving second connecting means, and wherein the first attaching holes or the second attaching holes are implemented as oval holes.

13. The medical implant according to claim 12, wherein a longitudinal axis of the oval holes is substantially oriented along a pelvic ring direction.

14. The medical implant according to claim 1, wherein the connecting arm comprises a lower rigidity than the posterior plate and the cranial plate, so that an alignment of the posterior plate can be adjusted relative to the cranial plate by plastically deforming the connecting arm.

15. The medical implant according to claim 1, wherein the cranial plate is convex in a transversal plane, and/or wherein the posterior plate is convex in the transversal plane.

16. The medical implant according to claim 1, wherein a first implant segment for attaching to the first pubic bone and a second implant segment for attaching to the second pubic bone are implemented substantially mirror-symmetrical to each other.

17. The medical implant according to claim 1, wherein a length of the posterior plate and a length of the cranial plate are substantially identical.

18. The medical implant according to claim 1, wherein the at least one connecting arm is disposed spaced apart in a pelvic ring direction from ends of the posterior plate and/or ends of the cranial plate.

19. A method for osteosynthesis of a symphysis rupture on a training body for training purposes by means of a medical implant, preferably by means of a medical implant according to claim 1, comprising the steps:
placing a cranial plate of the medical implant on a first pubic bone of the training body from a cranial side;
placing a posterior plate of the medical implant on a first pubic bone of the training body from a posterior side;
attaching the cranial plate to the first pubic bone;
attaching the posterior plate to the first pubic bone;
adjusting a symphysis spacing between the first pubic bone and the second pubic bone;
attaching the cranial plate to the second pubic bone and attaching the posterior plate to the second pubic bone-after adjusting the symphysis spacing.

20. The method according to claim 19, further comprising the step:
placing a positioning protrusion of the medical implant on the first pubic bone from a side facing toward the symphysis of the training body,
wherein the placing of the positioning protrusion is performed before attaching the cranial plate and/or the posterior plate on the first pubic bone.
